(19) United States
(12) Patent Application Publication
(10) Pub. No.: US 2004/0142868 A1
(43) Pub. Date: Jul. 22, 2004

(54) Method of treating liver steatosis in a mammal

(76) Inventor: Mark W. Sleeman, Mahopac, NY (US)

Correspondence Address: REGENERON PHARMACEUTICALS, INC, 777 OLD SAW MILL RIVER ROAD, TARRYTOWN, NY, 10591, US

(21) Appl. No.: 10/758,783

(22) Filed: Jan. 16, 2004

Related U.S. Application Data
(60) Provisional application No. 60/441,464, filed on Jan. 21, 2003.

(51) Int. Cl. A61K 38/18

(52) U.S. Cl. 514/12

(57) ABSTRACT

Methods of treating, inhibiting, and/or ameliorating liver steatosis in a subject in need thereof, comprising adminstering an agent capable of activating a ciliary neurotrophic factor (CNTF) receptor.

### CROSS-REFERENCE TO RELATED APPLICATIONS

This application claims the benefit under 35 USC §119(e) of U.S. Provisional 60/441,464 filed 21 Jan. 2003, which application is herein specifically incorporated by reference in its entirety.

### REFERENCE TO SEQUENCE LISTING

This application refers to sequences listed in a Sequence Listing hereinto attached, which is considered to be part of the disclosure of the invention.

### BACKGROUND OF THE INVENTION

1. Field of the Invention

The field of the invention is related to treating liver steatosis with agents capable of activating the ciliary neurotrophic factor (CNTF) receptor. More specifically, the invention relates to treating liver steatosis in a subject suffering thereof with CNTF or CNTF variants such as, for example, Axokine®.

2. Description of Related Art

Gloaguen et al. (1997) Proc. Natl. Acad. Sci. USA 94:6456-6461 and U.S. Pat. No. 6,565,869 describe the use of ciliary neurotrophic factor (CNTF) for treatment of obesity, including obesity associated with diabetes. Ntambi et al. (2002) Proc. Natl. Acad. Sci. USA 99:11482-11486 describe reduced adiposity in mice targeted with disruption of stearoyl-CoA desaturate (SCD).

### SUMMARY OF THE INVENTION

Experiments described below show that treatment with a modified CNTF molecule correlated with decreased liver steatosis and improved liver function as determined by serum ALT/AST ratio as well as enhanced biochemical responsiveness of the liver to insulin (e.g. phosphorylation of IRS-1, recruitment of PI3-kinase, and Akt-kinase phosphorylation). These changes were accompanied by rapid alterations in hepatic gene expression caused by Axokine™, most notably in the reduced expression of stearyol-CoA desaturase-1 (SCD-1), a rate-limiting enzyme in the synthesis of complex lipids, and increased expression of carnitine palmitoyltransferase-1, a gene which promotes lipid oxidation.

Accordingly, the invention features a method of treating liver steatosis in a subject in need thereof, comprising administering an agent capable of activating the ciliary neurotrophic factor (CNTF) receptor. In specific embodiments, the agent capable of activating the CNTF receptor is CNTF or a modified CNTF capable of activating the CNTF receptor, for example, Axokine™. Additional CNTF variants are described in U.S. Pat. Nos. 5,349,056, 5,846,935, 6,472,178, 6,440,702, and 6,565,869, which publications are herein specifically incorporated by reference in their entirety.

Preferred embodiments of the invention are those wherein the agent is formulated with an acceptable pharmaceutical carrier suitable for administration via subcutaneous, intramuscular, intradermal, intraperitoneal, intravenous, intranasal, epidural, and oral routes.

Further embodiments include a method of treating liver steatosis with an agent capable of activating the CNTF receptor and a second agent capable of ameliorating diabetes, e.g., insulin. In this embodiment, the therapeutic method may allow a decreased amount of the second agent to be administered when administered in combination with an agent of the invention.

In specific embodiments, the invention provides methods for treating, ameliorating, or improving liver steatosis, wherein treatment results in one or more of improved liver function as determined by ALT/AST ratio, reduced stearyol-CoA desaturase-1 (SCD-1) gene expression or activity, enhanced the biochemical responsiveness of the liver to insulin, and/or reduced synthesis of complex lipids.

A subject suitable for treatment by the methods of the invention is a mammal, more preferably a human subject, suffering from or at risk from suffering from liver steatosis. Contributing factor may include obesity and/or alcohol abuse, and may be accompanied by increased fat deposition in the liver (steatosis), steatohepatitis, cirrhosis, and hepatocellular carcinoma (Tilg et al. (2000) Mechanisms of Disease 343:1467-1476).

Other objects and advantages will become apparent from a review of the ensuing detailed description.

### BRIEF DESCRIPTION OF THE FIGURES

FIGS. 1A-1C. Treatment with Axokine™ decreased body weight (FIG. 1a) and improves fasating glucose serum glucose (mg/dl) and insulin (ng/ml) levels. Results of an oral glucose tolerance test are shown in FIG. 1c. All data are expressed as the mean (n≧6)±SEM. ANOVA: % BW, P<0.001 for both C-0.1 and C-0.3; Tolerance Test, P<0.001; glucose P<0.001; Insulin P<0.05. *−difference from ad lib fed db/db vehicle and PF controls by Dunnett post-hoc test.

FIGS. 2A-2D. Male db/db mice treated with Axokine™ at 0.1 or 0.3 mg/kg/day for 10 days had reduced (FIG. 2A) epididymal white adipose tissue (EWAT) compared to pair-fed (PF) or Vehicle (V) and (FIG. 2B) reduced total liver weight. Liver glycogen content (FIG. 2C) was significantly reduced by pair feeding (PF), while liver function (FIG. 2D) as assessed by the ration of serum ALT/AST was significantly improved by both doses of Axokine. Each bar represents mean±s.e.m. of n=6-8 animals. ANOVA: EWAT, P<0.001; Liver, P<0.001; Glycogen P<0.001. *−difference from ad lib fed db/db vehicle control by Dunnett post-hoc test.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated by reference.

General Description

The invention is based in part on findings that administration of a CNTF variant results in a far greater improvement in body weight and diabetic parameters such as fasting glucose and insulin levels, oral glucose tolerance, triglycerides and non-esterified free-fatty acids than can be achieved by comparable food restriction. The increased weight loss resulting from Axokine™ treatment is correlated with increased energy expenditure. The increased insulin sensitivity induced by Axokine™ administration is correlated with decreased liver steatosis and improved liver function as determined by serum ALT/AST ratio as well as enhanced biochemical responsiveness of the liver to insulin. These changes are accompanied by rapid alterations in hepatic gene expression caused by Axokine™ administration, most notably in the reduced expression of stearoyl-CoA desaturase (SCD-1), a rate-limiting enzyme in the synthesis of complex lipids. In addition, increased expression of camitine palmitoyltransferase-1 (CPT-1), a gene that promotes lipid oxidation, is also observed following Axokine™ administration. Similar changes in hepatic gene expression, and consequent improvements in glucose and lipid metabolism, were not observed in pair-fed or weight-matched control mice. Taken together, these findings demonstrate that Axokine™ exerts metabolic effects that substantially contribute to the marked improvements in glucose and lipid homeostasis in diabetic mice, and which cannot be achieved by equivalent caloric restriction or weight reduction alone.

Pharmaceutical Compositions

The present invention also provides pharmaceutical compositions. Such compositions comprise a therapeutically effective amount of an active agent, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. In a specific embodiment, the composition comprises a combination of an agent of the invention and a second agent capable of ameliorating diabetes. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention which will be effective in the amelioration of type 2 diabetes can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Combination Therapies

In numerous embodiments, the fusion polypeptides of the present invention may be administered in combination with one or more additional compounds or therapies. For example, CNTF or a modified CNTF can be co-administered in conjunction with one or more therapeutic compounds. The combination therapy may encompass simultaneous or alternating administration. In addition, the combination may encompass acute or chronic administration.

Treatment Population

Hepatic steatosis, also termed fatty liver, may be caused by a number of factors, including long term consumption of alcohol, obesity, exposure to hepatotoxins, and infection. In many patients, a specific risk factor may not be identified. Methods for clinical identification of hepatic steatosis are known to those of skill in the art. Accordingly, the population of patients to be treated with the methods of the invention are clinically identified through standard tests of liver function.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Materials and Methods

Animals and Experimental procedures. Male C57BL/KS-Lep^{db} (db/db ) and non-diabetic littermate mice (obtained from Jackson Laboratories) were obtained at 7-8 weeks of age, and were housed in 12 h of light per day at 69-74° C. and 40-60% humidity. All experiments began at 10 weeks of age and all animal procedures were conducted in compliance with protocols approved by the Institutional Animal Care and Use Committee. Axokine™ is a recombinant variant of human CNTF (for a complete description, see U.S. Pat. No. 6,472,178). Axokine™ (0.1 and 0.3 mg/kg, s.c.) and vehicle (V; 10 mM Sodium Phosphate, 0.05% Tween 80, 3%o PEG 3350, 20% Sucrose pH 7.5) were injected daily for 10 days. For glucose tolerance testing, all animals were fasted for 16-18 hours before gavaging with a standard glucose bolus, as previously outlined (Tonra et al. (1999) Diabetes 48(3), 588-94). For assessment of insulin activation of signaling molecules, animals from long term dosing studies outlined above were anesthetized and a bolus of insulin (1 U/kg) was administered through the jugular vein and at the indicated times the liver was rapidly removed and frozen at −80° C. until it could be processed.

Serum chemistry and tissue analysis. Serum samples reported were taken between 10:00 and 12:00 h and analyzed for glucose, triglycerides and cholesterol utilizing the Monarch blood chemistry analyzer (Instrumentation Laboratory Company, Lexington, Mass.). NEFAs were analyzed with a diagnostic kit (WAKO, Richmond, Va.) and insulin levels by ELISA (Linco, St. Charles, Mo.). Tissue samples for histological analysis were taken from all mice at the conclusion of the experiments and fixed overnight in 10% buffered formalin. For H&E staining, tissue was embedded in paraffin, sections cut at approximately 6 mm, placed onto glass slides, deparaffinized with xylene and processed using standard methods. For analysis of endogenous lipids, frozen sections of liver were mounted on glass slides and stained with Oil red O. Liver glycogen was measured from frozen tissues by assaying for glucose after amyloglucosidase digestion, with a correction for non-glycogen glucose.

Tissue lysates and SDS-PAGE. Liver samples were separately homogenized on ice in buffer A (1% NP-40 buffer, 50 mM Hepes pH 7.4, 150 mM NaCl, 1 mM EDTA, 30 mM sodium pyrophosphate, 50 mM sodium fluoride, 0.5 mM sodium orthovanadate, 5 μg/ml aprotinin, 5 μg/ml leupeptin, 1 mM PMSF) and centrifuged for 10 min at 14,000 g. The supernatant was taken and protein level quantified (BCA protein assay, Pierce, Rutherford, Ill.) and either used for immunoprecipitation or equal amounts of protein resolved by SDS-PAGE (Novex, 8% precast gels). Proteins were transferred to nitrocellulose membranes to be blocked and then immunoblotted with phospho-specific Akt (Ser473) polyclonal antibodies (New England BioLabs, Beverley, Mass.). After secondary antibody incubation (Goat anti-Rabbit HRP conjugated, Boehringer Mannheim) detection was by an enhanced chemiluminescence detection system (Renaissance, Dupont NEN products).

Real Time PCR and Northern Blotting. Tissues were rapidly dissected and immediately frozen at −80° C. RNA was isolated using Tri-reagent (MRC, Cincinnati, Ohio). Tissue specific expression was analyzed in separate reactions using the Taqman (Applied Biosystems, Foster City, Calif.) real-time PCR chemistry and detection system (SCD-1 forward 5′-GGTACTACAAGCCCGGCCTC-3′ (SEQ ID NO:1), reverse 5′-AGCAGTACCAGGGCACCAGC -3′ (SEQ ID NO:2) SCD-1 probe 6-FAM-TGCTGATGTGCTTCATCCTGCCCA(SEQ ID NO:3); GPAT forward 5′-CAGACGAAGCCTTCCGACG-3′ (SEQ ID NO:4), GPAT reverse 5′-GACTTGCTGGCGGTGAAGAG-3′ (SEQ ID NO:5), GPAT probe 6-FAM-AGGCTGATTGCAAACCTGGCTGAGC-TAMRA (SEQ ID NO:6); CPT-1 forward 5′-CTGCAACTTTGTGCTGGCC-3′ (SEQ ID NO:7), reverse 5′-TTGAACAGCTTGAGCCTCTGC-3′ (SEQ ID NO:8) CPT-1 probe 6-FAM-TGATGGACCCCACAACAACGGCA (SEQ ID NO:9); PPARα-forward 5′-GCCGAGAAGACGCTTGTGG-3′ (SEQ ID NO:10), PPARα-reverse 5′-TCGGACCTCTGCCTCTTTGTC-3′ (SEQ ID NO:11), PPARα-probe 6-FAM-CAAGATGGTGGCCAACGGCGTC-TAMRA (SEQ ID NO:12); PPARγ-forward 5′-ATGCCATTCTGGCCCACC-3′ (SEQ ID NO:13), PPARγ-reverse 5′-GGAATGCGAGTGGTCTTCCATC-3′ (SEQ ID NO:14), PPARγ-probe 6-FAM-ACTTCGGAATCAGCTCTGTGGACCTCTCC-TAMRA (SEQ ID NO:15); UCP 2 forward 5′-TAGTGCGCACCGCAGCC-3′ (SEQ ID NO:16), UCP 2 reverse 5′-AGCTCATCTGGCGCTGCAG-3′ (SEQ ID NO:17), UCP 2 probe 6-FAM-CAGTACCGTGGCGTTCTGGGTACCATC-TAMRA (SEQ ID NO:18). Control genomic DNA was used as a standard to estimate copies of molecule per cell and all probes were run with a no-reverse transcriptase control for assessment of any genomic DNA contamination. Samples were done in duplicate from pools of 3 individual animal samples. Results are expressed as fold change from vehicle treated db/db levels. Northern blots were done on samples from pools of 3, as described previously (Lambert et. al. (2001) Proc. Natl. Acad. Sci. USA, 98, 652-4657).

Indirect Calorimetry. Metabolic measurements were obtained using an Oxymax (Columbus Instruments International Corp., Columbus, Ohio) open circuit indirect calorimetry system. The system was calibrated against standard gas mixture to measure O₂ consumed (ml/kg/h) and CO₂ generated (ml/kg/hr). Energy expenditure (or heat) was calculated as the product of calorific value of oxygen (=3.815+1.232×respiratory quotient) and the volume of O₂ consumed. These measurements were taken on animals that had received 9 days of Axokine™ or vehicle treatment. The first 2 h of measurements was used as a period of adaptation for the animals and metabolic rate and activity were evaluated for a 24 hr period.

Statistical analyses. Data is expressed as mean±s.e.m. and analysis of variance (ANOVA) conducted using the program STATVIEW. When a significant F ratio was obtained (significance P<0.05), post hoc analysis was conducted between groups using a multiple comparison procedure with Bonferroni/Dunn correction of means (ANOVAᵣₘ) or Dunnett post hoc comparison. P-values less than P<0.05 were considered significant.

### Example 2

Dose Dependent Effect of Axokine™ on Body Weight.

To further explore the effects of Axokine™ on glucose and lipid metabolism, studies were conducted in C57BL/KS-Lep^{db} (db/db) mice, a murine model of type 2 diabetes that results from loss of functional leptin receptors (ObR's), and is thus leptin-resistant. In this strain of mice, the metabolic abnormalities manifest early during development, and are quite severe in young adult animals. Moreover, once established, these metabolic changes are resistant to modulation by caloric restriction or weight reduction, compared to other mouse models of obesity-associated insulin-resistance and dyslipidemia (Tonra et al. (1999) Diabetes 48(3), 588-94).

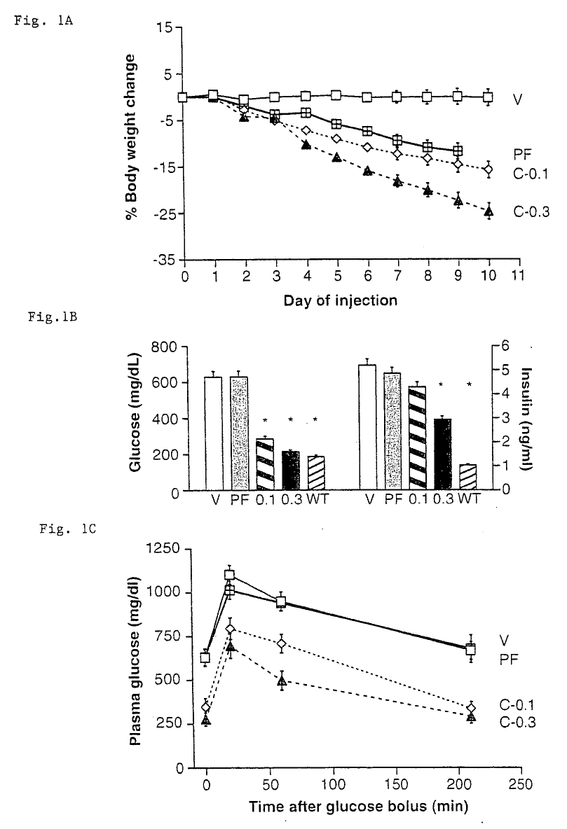

Groups of db/db mice received a daily subcutaneous injection of vehicle (V), Axokine™ at 0.1 or 0.3 mg/kg/day (C-0.1 or C-0.3) or were provided with the same amount of chow eaten by the C-0.3 treatment group (PF). Results are shown in FIGS. 1A-C.

Administration of Axokine™ to 10 week-old diabetic db/db mice decreased body weight in a dose dependent fashion (FIG. 1A). Even though this weight loss was associated with a dose-dependent decrease in food intake, matching caloric intake in a cohort of control db/db mice (“pair-fed”; PF) did not result in an equivalent weight loss. This is in contrast to observations made in other models of obesity, such as ob/ob mice or diet induced obesity (DIO), where little or no difference in weight loss is evident between CNTF-treated mice and PF controls.

Vehicle treated db/db mice (V) exhibited the fasting hyperglycemia (630±50 mg/dL), hyperinsulinemia (5.2±0.75 ng/ml) and impaired glucose tolerance characteristic of this strain. Fasting plasma glucose and insulin levels were significantly reduced in mice treated with Axokine™ (FIG. 1b), and oral glucose tolerance was also markedly improved (FIG. 1C; ANOVA, for both group and interaction, P<0.001). Similar improvements in glucose and insulin homeostasis were not seen in food-restricted control mice, matched for either equivalent caloric intake (c.f. C-0.3, animals receiving 0.3 mg/kg/d Axokine™) or body mass (c.f. C-0.1, animals receiving 0.1 mg/kg/d Axokine). Serum non-esterified free fatty acids (NEFAs) and triglycerides were also significantly reduced by Axokine™ relative to levels evident in both V and PF control mice (see Table 1).

TABLE 1
Effect of Treatment with Axokine ™

| | | db/db | | | |
|---|---|---|---|---|---|
| Parameter | db/? | Vehicle | C-0.1 | C-0.3 | PF |
| No. and sex | 24 males | 12 males | 24 males | 24 males | 18 males |
| Body Weight (g) | 26.4 ± 0.4* | 40.0 ± 0.6 | 35.3 ± 0.4* | 31.5 ± 0.6* | 35.7 ± 0.5* |
| Insulin (ng/ml) | 1.0 ± 0.2* | 5.2 ± 0.8 | 4.3 ± 0.4 | 2.9 ± 0.4 | 4.9 ± 0.4 |
| Glucose (mg/dl) | 185 ± 8* | 630 ± 50 | 285 ± 32* | 213 ± 23* | 631 ± 44 |
| NEFA (mmol/l) | 0.88 ± 0.05* | 1.53 ± 0.09 | 1.00 ± 0.1* | 1.05 ± 0.07* | 1.22 ± 0.04 |
| Triglyceride (mg/dl) | 69 ± 4* | 92 ± 10 | 70 ± 6 | 57 ± 3* | 73 ± 3 |
| Cholesterol (mg/dl) | 91 ± 5 | 121 ± 14 | 115 ± 5 | 89 ± 7* | 92 ± 3* |

### Example 3

Effect of Axokine™ on Non-Fasting Serum Glucose Levels.

In an experiment conducted on a separate cohort of mice, the effect of Axokine™ on non-fasting serum glucose levels was apparent within 2 days of the initiation of treatment, with a 50% reduction evident by day 4 (P <0.05), and reaching near non-diabetic control levels by day 10 (213±23; not significantly different from lean db/? controls, 185±8 mg/dL). At no time were any of the Axokine™-treated db/db mice hypoglycemic. Caloric restriction did produce a modest (˜25%) reduction in serum cholesterol levels, similar to that observed with Axokine™ treatment. However, food-restriction alone again failed to improve glycemic control in db/db mice. This observation is particularly significant, as rigorous control of hyperglycemia in diabetic humans can significantly attenuate the development of chronic complications associated with type-2 diabetes such as retinopathy and nephropathy (Colagiuri et al. (2002) Diabetes Care 25:1410-7); Klein et al. (1998) Diabetes Care 21 Suppl 3:C39-43).

### Example 4

Effect of Axokine™ Treatment on Epididymal White Adipose Tissue.

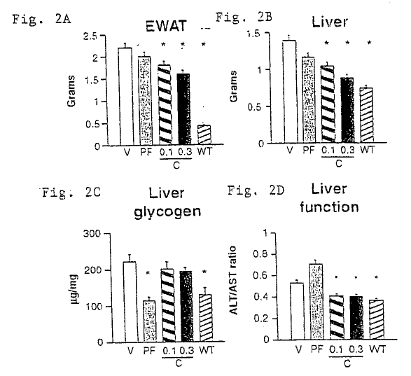

The effect of Axokine™ treatment on epididymal white adipose tissue (EWAT). The mass of this fat depot was reduced significantly in mice treated with Axokine (FIG. 2A) compared to mice injected with vehicle. Pair-feeding tended to reduce EWAT weight, though not to the same degree as that seen in Axokine™ treated mice. Microscopic evaluation revealed no obvious changes in adipocyte cell morphology in the EWAT of Axokine™ treated mice relative to PF controls, and there was no evidence of multi-locular cells type of the development of brown adiposities.

### Example 5

Measurement of Serum and Tissue Markers of Hepatic Function.

Serum and tissues markers of hepatic function were measured as described above. H &E staining of liver sections revealed reduced lipid vacuoles, which was confirmed by Oil Red-O staining which showed a clear reduction in the deposition of neutral lipids in CNTF treated groups, compared to both pair-fed and vehicle injected controls. Characteristically, db/db mice present with moderate hepatomegaly (FIG. 2B) and impaired liver function as indicated by an elevation in the serum alanine aminotransferase (ALT) and aspartate aminotransferase (AST) (FIG. 2D). These changes are thought to be secondary to hepatic accumulation of fat, which is evident in hematoxylin and eosin and Oil Red 0 stained sections of the livers of control db/db mice. Treatment of db/db mice with Axokine™ reduced the liver weight and normalized the serum ALT/AST. Lipid deposition in liver was also markedly reduced. In contrast, caloric restriction produced only a small, non-significant reduction in liver weight, which was accompanied by a marked depletion of hepatic glycogen stores. Caloric restriction did not improve hepatic function (ALT/AST) or decrease lipid deposition in the liver. The combined effect of Axokine™ to reduce serum glucose and lipid levels, preserve liver glycogen, reduce the ALT/AST ratio as well as the deposition of neutral lipid in the liver, are indicative of a marked improvement in obesity-related fatty liver that is distinct from the effects of food restriction or weight reduction alone.

### Example 6

Axokine™ Treatment Restores Hepatic Insulin Sensitivity.

After 10 days of Axokine™ treatment as outlined above, mice were anaesthetized, and an i.v. bolus of insulin or vehicle was administered. The liver was removed and frozen in liquid nitrogen 2 minutes later. Equal amounts of liver homegenate were immunoprecipitated with anti-IRS-i and blotted with anti-phosophotyrosine (pTyr) or antibodies against the p85 subunit of P13-kinase (p85). Comparison of the signals obtained showed rapid tyrosine phosphorylation of IRS-1 and the recruitment of the p85 subunit of PI3-kinase in lean non-diabetic mice and a blunted response in the vehicle treated db/db mice state. In contrast, robust tyrosine phosphorylation of IRS-1 and subsequent p85 recruitment was seen consistently in both groups of Axokine™ treated mice (C-0.1 and C-0.3), but not pair-fed mice. Activation of the downstream signaling molecule Akt-kinase was assessed with phospho-specific antibodies of lysates taken from similarly treated mice 10 min after insulin administration. A consistent increase in phospho-Akt was observed in lean control mice, but not in diabetic mice that were injected with vehicle or pair-fed. In contrast, the insulin stimulated increase in phospho-Akt was at least as robust in Axokine™-treated db/db mice as in lean, non-diabetic littermates. Moreover, a clear decrease in basal levels of phospho-Akt was evident in PF mice, and this was also corrected by administration of Axokine™.

### Example 7

Evaluation of Differential Changes in Gene Expression.

To elucidate the molecular mechanism by which Axokine™ induces the physiological changes described supra, differential changes in gene expression were probed by northern blot analysis as well as real time PCR assays (Taqman) in hepatic tissue as follows. Liver tissue was collected from male db/db mice treated with Axokine (C-0.3) for 4 days and from pair-fed or vehicle-injected controls. Total RNA was prepared for assessment by Northern blots for stearoyl-CoA desaturase (SCD-1), glycerol-palmitoyl acyl-transferase (GPAT), CPT-1 and UCP-2 & PPARa PPARg, phosphoenolpyruvate carboxylase (PEPCK) and acyl CoA oxidase (ACO). Each lane represents a pool of RNA from 3 mice. The same RNA sample were analyzed by real time PCR and results are expressed as bar graphs as the fold increase/decrease relative to controls that received only vehicle injections (mean±SEM n=6-10 individual samples). ANOVA: CPT-1 P<0.001; PPARg ,P<0.001; GPAT P<0.001. *−difference from ad lib fed db/db vehicle control by Dunnett post-hoc test. One of the most striking features of Axokine™ treatment was the specific decrease in the expression of genes associated with triacylglycerol synthesis and uptake in liver, such as GPAT and SCD-1; the changes in SCD-1 in particular were far more impressive than those noted with PF alone. Associated with treatment, but also evident in the PF group, is an increase in PPARα mRNA (<2 fold), together with its target enzymes of FA oxidation, CPT-1 and UCP-2. No change could be could be detected in ACO mRNA, however there may be subtle changes in activation of this enzymes that may further contribute to increased b-oxidation. The net effect of these changes would be to reduce fatty acid biosynthesis and decrease hepatic lipid content, consistent with the above histological and serum chemistry results. The expression of several genes important in carbohydrate metabolism and known to be regulated by leptin, such as PEPCK, glucose-6-phosphatase, fructose bisphosphatase and hexokinase, were not altered by Axokine™ treatment. Analysis of EWAT showed Axokine™ administration produced a rapid, two-fold decrease in mRNA for fatty acid synthase (FAS) in EWAT, but surprisingly no changes in expression in ACC, GPAT, PPARγ UCP-1, UCP-2, UCP-3, GLUT4, GLUT1 and CPT-1 could be detected in EWAT by RT-PCR.

These findings suggest that, in addition to its established appetite-suppressing actions in the hypothalamus, Axokine™ acts to decrease the synthesis and increase the oxidation of lipids in the livers of db/db mice. Here, the specific Axokine™-mediated reduction in the expression of hepatic SCD-1 is particularly noteworthy. Recently, Cohen et al. (2002) Science 297:240-243) reported that leptin administration dramatically reduced SCD-1 expression in the livers of ob/ob mice. Moreover, mice deficient in SCD-1 were lean and hyper-metabolic, while ob/ob mice bearing a mutation in the SCD-1 gene were less obese and exhibit elevated levels of energy expenditure compared to control ob/ob mice. The livers of these mice were histologically normal, and triglyceride stores were much reduced. These effects of leptin in ob/ob mice closely parallel the effects of Axokine™ on liver structure and function observed here in obese, diabetic db/db mice, suggesting that Axokine™ might also exert a positive effect on energy expenditure that could account for the differential loss in body weight seen in treated mice compared to pair-fed controls.

### Example 8

Assessment of Effects of Axokine™ on Energy Expenditure.

Groups of db/db mice received daily subcutaneous injections of vehicle or Axokine™ (0.3 mg/kg, C-0.3) for 9 days. Indirect calorimetry was performed over a 24 hour period following the last injection. Oxygen consumption (VO₂; ml/kg/hr); carbon dioxide production (VCO₂; ml/kg/hr), energy expenditure as heat (kcal/hr), and locomotor activity was measured for C-0.3 and V treated groups in at approximately 1 hr intervals. ANOVA: oxygen, P<0.001; carbon dioxide, P <0.001; energy P<0.001. Control and Axokine™ treated mice were evaluated by indirect calorimetry to assess the effects of Axokine™ administration on energy expenditure. In control db/db mice, metabolic rate is increased during the dark period (night), as indicated by increased oxygen consumption and carbon dioxide production. These parameters are reduced in the subsequent light period, when the animals are normally at rest. Control mice also showed an increase in heat production during the dark period, reflecting the combined effects of increased physical activity and/or thermogenesis. Heat production declines in the lights-on period (day) when the animals are at rest, and thus reflects basal energy expenditure. Treatment of db/db mice with Axokine induced an increase in metabolic rate, as evidenced by elevations in VO₂ and VCO₂ and increased energy expenditure relative to controls, particularly during the light period. Locomotor activity was characteristically higher during the dark period than during the light period in control db/db mice, and neither the pattern nor overall level of activity was altered by Axokine™ treatment, indicating that the observed increase in energy expenditure was not secondary to increased physical activity. Axokine™ treatment did not induce a switch in metabolic substrate (i.e. a differential respiratory quotient or VCO₂/VO₂ ratio). These observations show that Axokine™ treatment can produce a differential reduction in body weight in obese and diabetic (db/db) mice by decreasing appetite while maintaining or increasing energy expenditure, as is the case for leptin treatment in the leptin-deficient ob/ob mice. That Axokine™ treatment also reduces hepatic expression of SCD-1 supports the proposal that this enzyme plays a key role in mediating the pro-metabolic effects of these both of these proteins.

We claim:

1. A method of treating liver steatosis in a subject in need thereof, comprising administering an agent capable of activating a ciliary neurotrophic factor (CNTF) receptor.

2. The method of claim 1, wherein the agent capable of activating the CNTF receptor is CNTF or a modified CNTF.

3. The method of claim 2, wherein the modified CNTF is Axokine™.

4. The method of claim 1, wherein treatment results in one or more of improved liver function as determined by ALT/AST ratio, reduced stearyol-CoA desaturase-1 (SCD-1) gene expression or activity, enhanced the biochemical responsiveness of the liver to insulin, and/or reduced synthesis of complex lipids.

5. The method of claim 1, wherein liver steatosis results from alcohol consumption, obesity, and/or exposure to a hepatotoxin.

6. A method of improving liver steatosis in a subject in need thereof, comprising administering to the subject an agent capable of activating the CNTF receptor complex such that liver steatosis is improved.

7. The method of claim 6, wherein the agent capable of activating the CNTF receptor is CNTF or a modified CNTF.

8. The method of claim 7, wherein the modified CNTF is Axokine™.

9. The method of claim 6, wherein treatment results in one or more of improved liver function as determined by ALT/AST ratio, reduced SCD-1 gene expression or activity, enhanced the biochemical responsiveness of the liver to insulin, and/or reduced synthesis of complex lipids.

10. The method of claim 6, wherein liver steatosis results from alcohol consumption, obesity, and/or exposure to a hepatotoxin.
